(19) United States
(12) Patent Application Publication
(10) Pub. No.: US 2010/0274276 A1
(43) Pub. Date: Oct. 28, 2010

(54) ANEURYSM TREATMENT SYSTEM, DEVICE AND METHOD

(76) Inventors: Ricky Chow, Minneapolis, MN (US); John Schreiner, Minneapolis, MN (US); William K. Durfee, Edina, MN (US); John M. Zimmerman, Chanhassen, MN (US)

Correspondence Address: WHYTE HIRSCHBOECK DUDEK S.C.;INTELLECTUAL PROPERTY DEPARTMENT, 33 East Main Street, Suite 300, Madison, WI, 53703-4655, US

(21) Appl. No.: 12/427,910

(22) Filed: Apr. 22, 2009

(51) Int. Cl. A61F 2/01

(52) U.S. Cl. 606/200

(57) ABSTRACT

An occlusion system, method, and apparatus which treats an aneurysm in a primary vessel such as an intracranial vessel. Aneurysm types treatable by means of this invention include bulge (sometimes referred to as a true aneurysm), dissecting aneurysms in which the layers of vessel wall are partially delaminated, and what are referred to a “false” aneurysm defined by a relatively small opening in the parent vessel wall fluridically coupled to a bulb-shaped sac, also defined by the vessel wall. The system includes an occlusion device configured for deployment in the parent vessel that restricts fluid flow to the aneurysm. A patch on the device covers the neck of the aneurysm. The system includes a marker in a defined relationship with the patch, which allows for axial and rotational positioning of the device.

### FIELD OF THE INVENTION

The present invention relates to a system, apparatus or device and method for treating an aneurysm. In particular, embodiments of the present invention relate to an occlusion system, capable of being deployed in the intracranial vasculature, for at least in part containing an aneurysm and for treating the same.

### BACKGROUND OF THE INVENTION

Several methods of treating aneurysms are in use clinically, with varying degrees of success. For example, open craniotomy is a procedure by which an aneurysm is located, and treated, extravascularly. This type of procedure has significant disadvantages. For example, the patient undergoing open craniotomy must undergo general anesthesia. Also, the patient undergoes a great deal of trauma in the area of the aneurysm by virtue of the fact that the surgeon must sever various tissues in order to reach the aneurysm. In treating cerebral aneurysms extravascularly, for instances, the surgeon must typically remove a portion of the patient's skull, and must also traumatize brain tissue in order to reach the aneurysm.

Other techniques used in treating aneurysms are performed endovascularly. Such techniques typically involve attempting to form a mass within the sac of the aneurysm. Typically, a microcatheter is used to access the aneurysm. The distal tip of the micro catheter is placed within the sac of the aneurysm, and the microcatheter is used to inject embolic material into the sac of the aneurysm. The embolic material includes, for example, detachable coils. The injection of these types of embolic materials suffers from disadvantages, most of which are associated with migration of the embolic material out of the aneurysm into the parent artery. This can cause permanent and irreversible occlusion of the parent artery.

Another endovascular technique for treating aneurysms includes the use of a device to restrict the flow of blood through the neck of an aneurysm. Such a device is deployed endovascularly. Aneurysms are often located in close proximity to, or adjacent to collateral and side branching vessels. Aneurysms often form in close proximity to vessels, will be occluded by treating the aneurysm in this manner. Therefore, the ability to place the device accurately is critical to successful treatment. It would be advantageous for such an endovascular device to be accurately deployed so as to cover only the aneurysm and have limited interference with vessels adjacent or collateral to the targeted aneurysm.

### BRIEF SUMMARY OF THE INVENTION

Briefly in one aspect, the invention includes a cylindrical intravascular aneurysm treatment device. The device includes an expandable sleeve or scaffold having a fluid-flow-restricting portion or patch, where the patch is located on the sleeve so as to inhibit fluid flow through at least part of the sleeve when the sleeve is intravascularly deployed. The device also includes a radiopaque marker, or a plurality of radiopaque markers, disposed on the sleeve, the marker(s) having a defined relationship with respect to the patch such that deployment of the sleeve within the vasculature while observing the marker(s), e.g. by means of a fluoroscope, permits precise orientation of the patch.

In another aspect, the invention includes an intracranial aneurysm treatment device with an expandable scaffold. The scaffold includes a fluid-flow-restricting patch that inhibits fluid flow through a non-circumferential portion of the scaffold. Furthermore, the scaffold includes a radiopaque marker having a non-symmetrically or asymmetrically defined relationship with respect to the patch. The marker is configured to be observed during deployment of the scaffold within the cranial vasculature for determining the location of the patch.

In yet another aspect, the invention includes a method of treating an aneurysm, preferably an intracranial aneurysm. Steps of the method include providing an aneurysm treatment device comprising an expandable scaffold, in combination with a fluid-flow-restricting, non-circumferentially positioned patch and a radiopaque marker having a defined relationship with respect to the patch. An additional step in the method includes deploying the device into the vasculature of a patient with an aneurysm while simultaneously monitoring the location of the radiopaque markers to place the patch adjacent the aneurysm in a location which restricts fluid flow thereto. The method also includes permitting the aneurysm to heal with restricted body fluid flow thereto.

In one aspect, the healing step is accomplished in the absence of aneurysm-filling embolic materials such as polymers, metals of various sorts and various states or other embolic materials known to the art.

In another aspect, the invention includes an aneurysm delivery system. An aneurysm treatment device comprising an expandable scaffold, in combination with a fluid-flow-restricting, non-circumferentially positioned patch and a radiopaque marker having a defined relationship with respect to the patch; and means for deploying the aneurysm treatment device into a patient's vasculature so that fluid flow to the aneurysm is restricted.

In another aspect, the invention includes an intracranial aneurysm treatment device with an expandable scaffold. The scaffold includes a fluid-flow-restricting patch that inhibits fluid flow through a non-circumferential portion of the scaffold. Furthermore, the scaffold includes an echogenic marker having a non-symmetrically or asymmetrically defined relationship with respect to the patch. The marker is configured to be observed during deployment of the scaffold within the cranial vasculature for determining the location of the patch.

### BRIEF DESCRIPTION OF THE FIGURES

The invention will now be exemplified with the following Figures, Detailed Description and attached Claims. It is understood that some of the Figures are an inventor's visualization of the structures and relationships shown as a combination of a visual impression and what would be seen, e.g. by viewing the device with a fluoroscope. In other words, once the device is intravascularly placed, visual monitoring is no longer possible and the fluoroscopically viewable features, e.g., the markers, are all that can be monitored.

FIG. 1A is a perspective view of an aneurysm treatment device in accordance with at least one embodiment of the present invention;

FIG. 1B is a longitudinal end view of the device in FIG. 1A in accordance with at least one embodiment of the present invention;

FIG. 2A is a perspective view of a sleeve-shaped scaffold for use in an aneurysm treatment device in accordance with at least one embodiment of the present invention;

FIG. 2B is a flat perspective view of the scaffold in FIG. 2A;

FIG. 3A is a perspective view of an aneurysm treatment device having an internally placed patch in accordance with at least one embodiment of the present invention;

FIG. 3B is a longitudinal end view of the device in FIG. 3A in accordance with at least one embodiment of the present invention;

FIG. 4A is a perspective view of an aneurysm treatment device having a pair of patches in accordance with at least one embodiment of the present invention;

FIG. 4B is a longitudinal end view of the device in FIG. 4A in accordance with at least one embodiment of the present invention;

FIG. 5A is a perspective view of an aneurysm treatment device having a patch integrally formed with a scaffold in accordance with at least one embodiment of the present invention;

FIG. 5B is a longitudinal end view of the device in FIG. 1A in accordance with at least one embodiment of the present invention;

FIG. 6 is a perspective view of an aneurysm treatment device having a patch integrally formed with the scaffold establishing the shape of the patch in accordance with at least one embodiment of the present invention;

FIG. 7A is a graphical depiction of an aneurysm within a cerebral artery;

FIG. 7B is a graphical depiction of an aneurysm treatment device deployed at an aneurysm depicted in FIG. 7A;

FIGS. 8A-C are graphical depictions of an aneurysm treatment device deployed at the site of aneurysm, including a acute, intermediate, and long term treatment time frame;

FIG. 9 represents a cross sectional depiction of a cerebral artery aneurysm site with an aneurysm treatment system deployed;

FIG. 10A is an uncut implant frame with radiopaque markings in accordance with at least one embodiment of the present invention;

FIG. 10B is a perspective view of an aneurysm treatment device having radiopaque markers on the top side in accordance with at least one embodiment of the present invention;

FIG. 10C is a representation of an aneurysm treatment device correctly placed within a vessel in accordance with at least one embodiment of the present invention;

FIG. 10D is an alternative perspective view of the representation in FIG. 10C;

FIG. 10E is a representation of an aneurysm treatment device incorrectly placed within a vessel in accordance with at least one embodiment of the present invention;

FIG. 10F is an alternative perspective view of the representation in FIG. 10E;

FIG. 11A is an uncut implant frame with radiopaque markers in accordance with at least one embodiment of the present invention;

FIG. 11B is a perspective view of an aneurysm treatment device having radiopaque markers on two sides in accordance with at least one embodiment of the present invention

FIG. 12A is an uncut implant frame with radiopaque markers in accordance with at least one embodiment of the present invention;

FIG. 12B is a perspective view of an aneurysm treatment device having radiopaque markers on the top side and a projection extending towards a medial point of the patch in accordance with at least one embodiment of the present invention;

FIG. 12C is a perspective view of a graphical representation of a correctly deployed device having the configuration of 12A;

FIGS. 12D and 12E are alternative perspective views of the representation in FIG. 12C.

FIG. 13A is an implant frame with radiopaque markers in accordance with at least one embodiment of the present invention;

FIG. 13B is a perspective view of an aneurysm treatment device having radiopaque markers on the top side and a plurality or projections extending towards to the edges of the patch in accordance with at least one embodiment of the present invention;

FIG. 14A is a perspective view of an aneurysm treatment device having a marker extending around the periphery of the patch in accordance with at least one embodiment of the present invention;

FIG. 14B is a perspective view of a graphical representation of a correctly deployed device having the configuration of FIG. 14A;

FIG. 14C is a side view graphical representation of a correctly deployed device having the configuration of FIG. 14A;

FIG. 15 is a perspective view of an aneurysm treatment device having a marker covering the entire patch in accordance with at least one embodiment of the present invention;

FIG. 16A is a perspective view of an alternative marker configuration located on at least a portion of the scaffold in accordance with at least one embodiment of the present invention.

FIGS. 16B-E are various perspective and side views of a graphical representation of a deployed device having the configuration of FIGS. 16A; 16B and 16C are representations of a correctly placed device, while 16D and 16E are representations of incorrectly placed devices;

FIG. 17A is a perspective view of an alternative marker configuration located on at least a portion of the scaffold in accordance with at least one embodiment of the present invention;

FIG. 17B is a perspective view of an alternative marker configuration located on at least a portion of the scaffold in accordance with at least one embodiment of the present invention;

FIGS. 17C-F are various perspective and side views of a graphical representation of a deployed device having the configuration of FIGS. 17B, 17C and 17D are representations of a correctly placed device, while 17E and 17F are representations of incorrectly placed devices;

FIG. 18A is a perspective view of a mechanical interlock portion of a guide wire in accordance with at least one embodiment of the present invention;

FIG. 18B is a side view of the device attached to a mechanical interlock portion of a guide wire in accordance with at least one embodiment of the present invention;

FIG. 19A is a perspective view of a pre-deployed device on a mechanical interlocking guide wire absent a constraining catheter, or similar structure, keeping the device in a collapsed state in accordance with at least one embodiment of the present invention;

FIG. 19B is a perspective view of a partially deployed device in accordance with at least one embodiment of the present invention;

FIG. 19C is a perspective view of the device in FIGS. 19A and 19B fully deployed;

FIG. 20 is a perspective view of an alternative device configuration having a pilot indicator in accordance with at least one embodiment of the present invention;

FIG. 21A is a perspective view of a distal end of a crimped or collapsed delivery wire having a generally pointed pilot indicator in accordance with at least one embodiment of the present invention;

FIG. 21B is a perspective view of a distal end of an alternative configuration for both delivery wire and pilot indicator in accordance with at least one embodiment of the present invention;

FIGS. 21C (pre-deployment) and 21D (post-deployment) depict the same delivery wire configuration as 21B with an alternative configuration for the pilot indicator in accordance with at least one embodiment of the present invention;

FIG. 22 is a perspective view of a combination of the distal end of the delivery wire, implant, and pilot indicator of FIG. 21D;

FIGS. 23A and 23B depict an alternative embodiment of the delivery wire and device interlock mechanism in accordance with at least one embodiment of the present invention;

FIGS. 24A and 24B depict an alternative embodiment of the delivery wire and device interlock mechanism in accordance with at least one embodiment of the present invention;

### DETAILED DESCRIPTION OF SYSTEM, DEVICE, AND DELIVERY METHOD

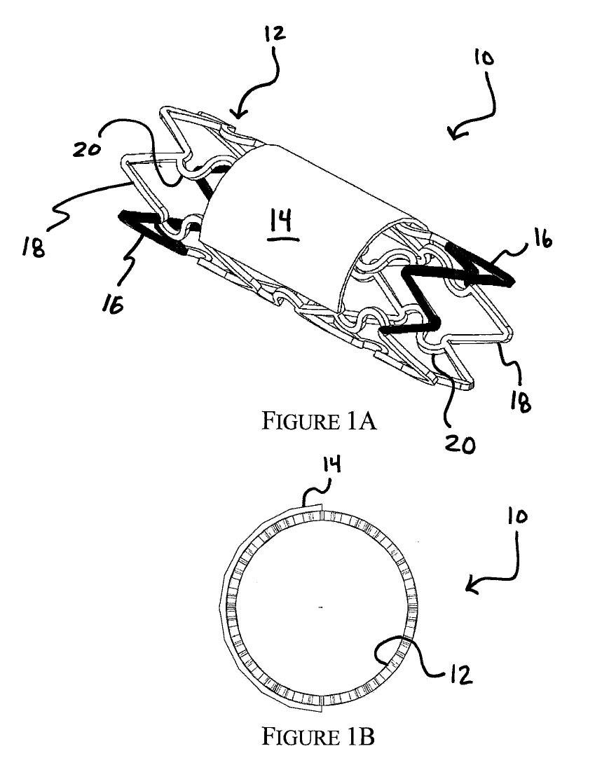

Briefly in one aspect and according to at least one embodiment of the present invention, an aneurysm treatment device 10 is provided in FIGS. 1, and 3-6. The device 10, also referred to as a selective occlusion device (SOD), is primarily designed to treat aneurysms by removing the aggravating forces that distend and damage the delicate vasculature, allowing the tissue to heal and remodel back to a non-diseased, non-distorted state. Aneurysm types treatable by means of various embodiments of the present invention include bulge aneurysms (sometimes referred to as a true aneurysm), dissecting aneurysms in which the layers of vessel wall are partially delaminated, and what are referred to a “false” aneurysm defined by a relatively small opening in the parent vessel wall fluidically coupled to a bulb-shaped sac, also defined by the vessel wall.

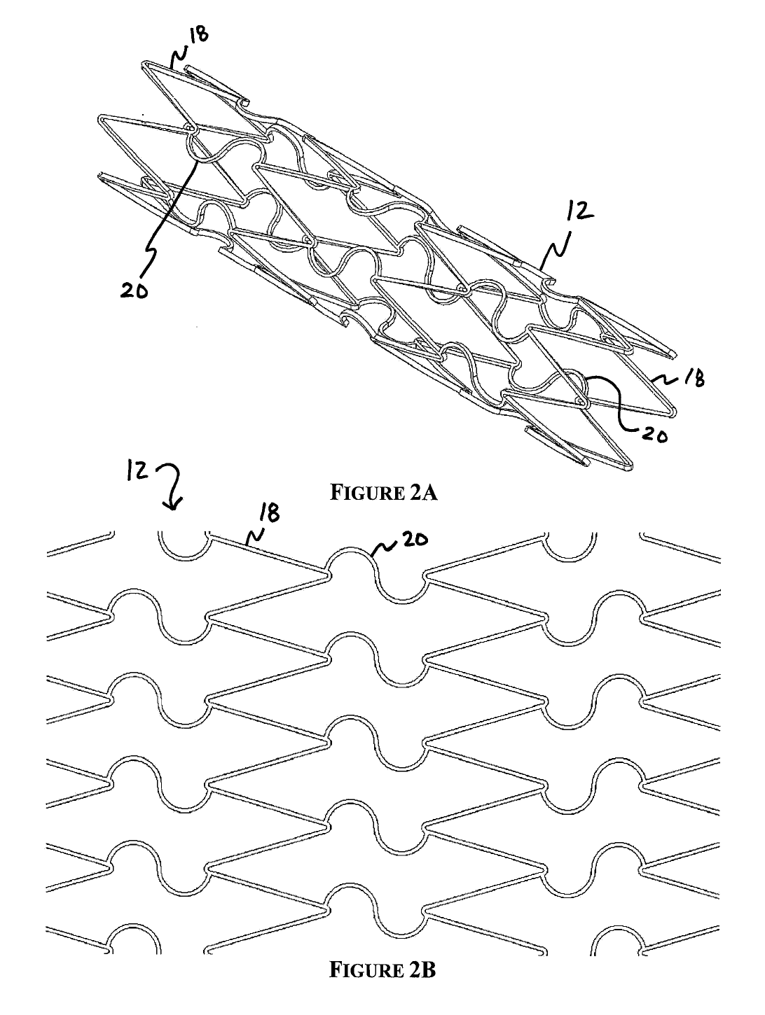

The device 10 includes a scaffold 12, a fluid-flow-restricting region or patch 14, and radiopaque markers 16. An exemplary scaffold 12, which is provided in FIGS. 2A and 2B, includes a plurality of serpentine ring segments 18. Segments 18 are connected to each other by at least one sigmoidal link 20. Various suitable exemplary embodiments of the scaffold 12 are described within U.S. Pat. Nos. 5,972,027 and 6,602,282, which are incorporated by reference in their entirety herein. A combination of structure of the Advantec Duraflex design, and the Scimed RADIUS design is also contemplated. The segments 18, 20 are radially expansible, which means that they can be converted from a small diameter configuration to a radially expanded, typically cylindrical, configuration. The expanded configuration is achieved when the prosthesis is implanted at the desired target site.

The device 10 is a self-expanding scaffold 12 designed with a minimum amount of outward radial force needed to anchor the device in place while supporting a membrane to disrupt flow across the opening to the aneurysm sac or cavity. The use of a self expanding material causes less damage to surrounding vessels during deployment, but also allows for the device 10 to be recaptured and repositioned as needed. The scaffold 12 is repositioned by holding a delivery wire steady and advancing the deployment catheter. In order to deploy the scaffold 12, the delivery wire is held steady and the catheter is pulled back. The delivery wire will be designed to maintain acquisition of the scaffold after less than 100% expansion. If at that time the healthcare professional, typically an interventional neuroradiologist, does not feel comfortable with the placement of the device in relation to the aneurysm or surrounding vessels, the scaffold can be pulled back into the catheter via the delivery wire. If the scaffold is expanded beyond its intended expansion state, then the scaffold cannot be retrieved.

The scaffold 12 is preferably a highly flexible support frame, which is advantageous for navigating through tortuous vasculature leading to e.g., an intracranial aneurysm. The preferred material is Nitinol® nickel titanium alloy. Alternative materials include stainless steel, tantalum, various forms of cobalt/chromium alloys, various polymers such as poly-lactic-co-glycolic acid (PLGA), polycaprolactone (PCL), polydoxanone (PDO) and various other suitable self-expanding, rigid, and/or flexible materials. Alternatively, the scaffold is formed from at least partially an echogenic material.

Cerebral vasculature is very delicate. Accordingly, the device 10 is delivered through a mechanism that places minimal pressure upon the vessel wall. The outward radial force can be carefully controlled, and thereby minimizes damage to the vessel walls. Furthermore, the device 10 configuration allows fluid access to vessels nearby or adjacent to the aneurysm, thereby allowing blood flow to critical brain tissues. Accurate placement of the patch 14 at the vessel opening, or access port of the aneurysm, is achieved through in-situ rotation, which is guided by radiopaque markers 16 viewed through a fluoroscope. Alternatively, echogenic markers can be used and viewed ultrasonically.

Referring to FIGS. 2A and 2B, the scaffold configuration is provided with a closed-cell design. The scaffold 12 has a low profile, which is beneficial for minimized disruption of blood flow within the vessel. The device 10 is manufactured in varying sizes, which are based upon the interior circumference of the vessel. Depending upon the location of the aneurysm, the vessel size can vary significantly. Smaller sizes are particularly useful for treating intracranial aneurysms. For purposes of the present invention, intracranial aneurysms include aneurysms located within the vasculature superior to the aortic arch, which includes the carotid arteries. The circumference post deployment ranges from about 2.0 mm to about 6.0 mm. It is conceivable that the post deployment circumference can be less than about 2.0 mm or greater than about 6.0 mm depending upon the diseased vessel interior circumference. Length of the device 10 ranges from about 15 mm to about 20 mm. It is further contemplated that the device 10 length is less than about 15 mm or greater than about 20 mm. At full deployment the change in length of the scaffold 12 is between about 5% to about 25% the pre-deployed length. Length of the scaffold 12 preferably changes less than about 20% from the pre-deployed configuration.

The surface area of the fluid-blocking or fluid-flow restrictive patch 14 on the scaffold 12 is kept to a minimum, ideally spanning only the aneurysm opening or access region. Suitable patch materials include parylene, polysiloxane, silicone, polyurethane, PTFE, and other biostable polymers and materials. An appropriate material has long term blood or body fluid compatibility. A further characteristic of a viable patch material is that it is applied as a highly flexible membrane having a consistently controllable thickness strongly adhered to the frame to allow for loading, storage, and deployment without damage. Current techniques include electro-spinning, dip coating, casting, and others techniques generally known in the art. An additional benefit of a minimal non-circumferential patch 14 size is reduced scaffold 12 stiffness as compared to a circumferentially positioned patch, thereby enhancing scaffold 12 flexibility.

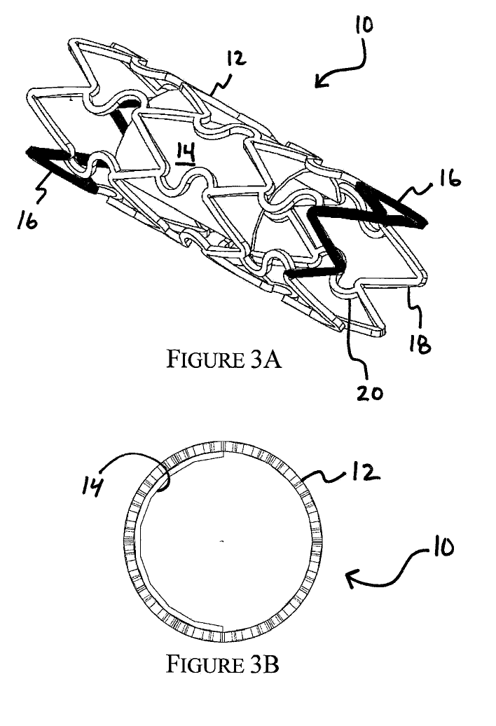

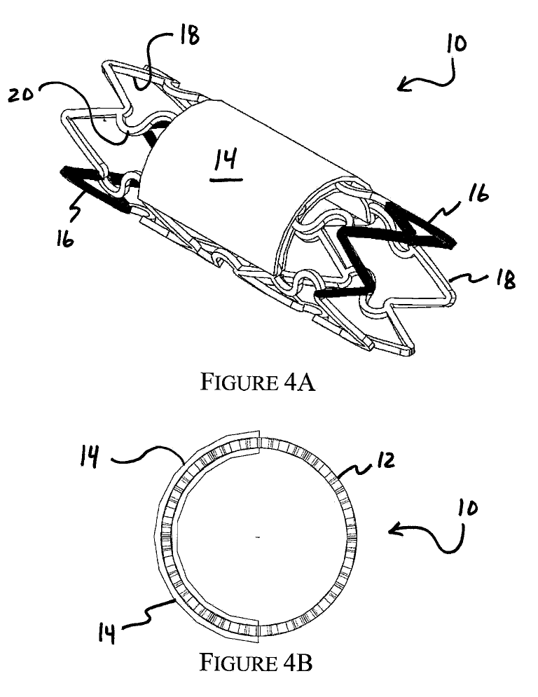

Referring to FIGS. 3-6, alternative embodiments of the SOD 10 are shown. Placement of the patch 14 with respect to the scaffold 12 can be changed. In at least one embodiment, the patch 14 is placed on the internal surface of the scaffold (FIGS. 3A and 3B). An alternative embodiment includes a pair of patches 14 (see FIGS. 4A and 4B). The pair of patches 14 are preferably uniform in shape, one being placed on the exterior of the scaffold, as shown in FIG. 1A, and the second being placed on the interior surface of the scaffold, as shown in FIG. 3A.

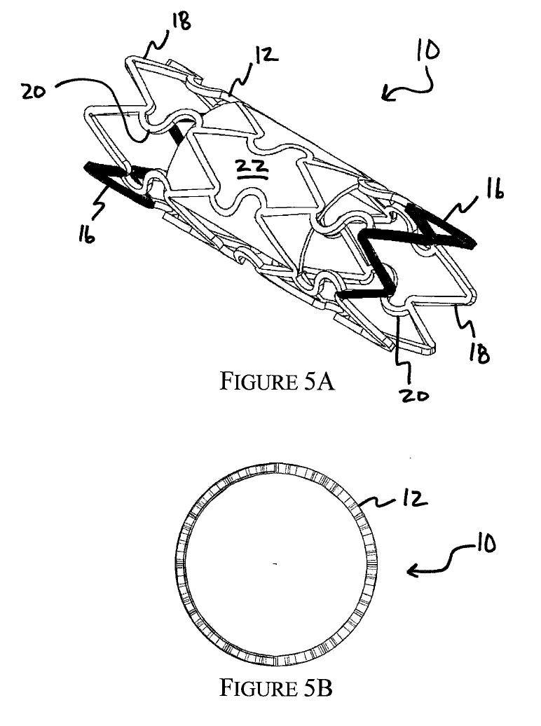

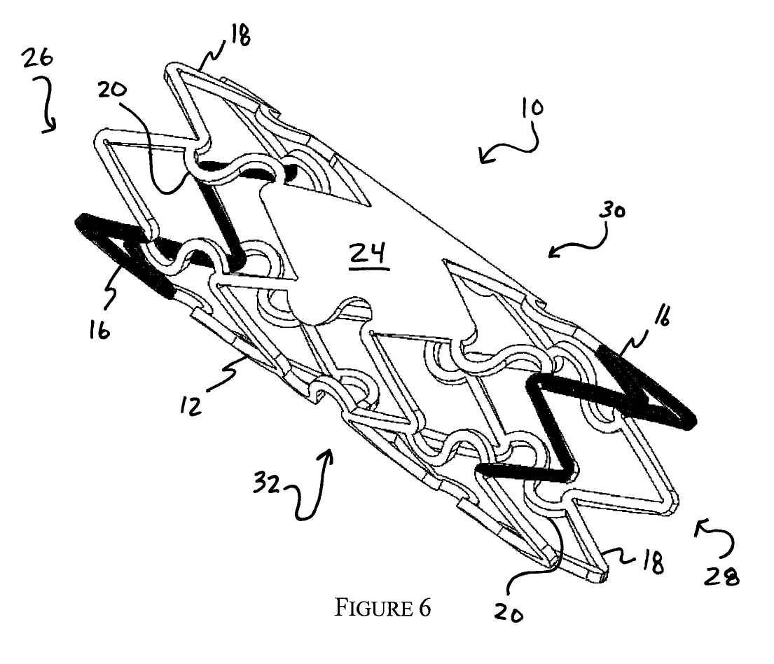

In yet another alternative embodiment (FIGS. 5A and 5B), a patch 22 is integrally formed with the scaffold 12, rather than placed on top of the scaffold as in previous embodiments. The patch 22 is formed from a biostable polymer and pressed into the desired location on the scaffold 12. The present embodiment has the advantage of limiting the draft thickness of the device 10, which can be seen in FIG. 5B. The patch 22 is formed at about the same or smaller thickness as the scaffold 12, thereby limiting the effect upon fluid flowing through the vessel. In another alternative embodiment (FIG. 6), a patch 24 is integrally formed with the scaffold 12. Shape of the patch 24 is restricted by the segments 18, 20.

A pair of radiopaque markers 16 is positioned at a proximal end 26 and distal end 28 of the aneurysm treatment device 10. The markers 16 are located on the device 10 for indicating rotational position of the membrane 14 during deployment. Platinum is embedded in the scaffold 12 at the proximal and distal ends 26, 28 acting as a fluoroscopic marker 16. A wide variety of methods for combining radiopaque materials with the scaffold 12 are contemplated, including alloy formation, plating, coating, physical vapor deposition, adhesive, and other suitable methods generally known in the art. However, due to the added stiffness to the design, thin layers of platinum are preferred for obtaining radiopacity. Alternatively, gold, tantalum, platinum, palladium, BaSO₄ or other noble and biocompatible radiopaque metals and metal alloys are suitable for use as markers 16. Alternatively, radiopaque coils can be attached to the scaffold for obtaining radiopacity. In the present embodiment, the markers are oriented for assisting rotational positioning while viewed through a fluoroscope, or alternative method of viewing in-situ radiopaque markers. The radiopaque material is placed on half of the exterior surface of the distal and proximal segments 18. The markers 16 are located at opposing ends 26, 28 and opposing sides 30, 32 of the device 10. The distal marker 16 is located at a top side 30, while the proximal marker 16 is located at a bottom side 32. Placement of the markers 16 represents a defined relationship with respect to the patch 14 such that deployment of the sleeve 10 within the vasculature determines the location of the patch 14. Alternative radiopaque configurations are contemplated, several of which are more fully described herein.

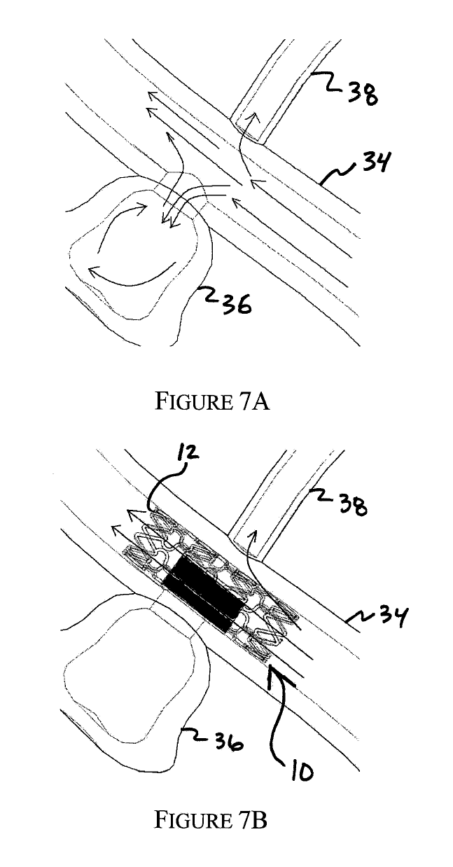

Referring to FIGS. 7A and 7B, an exemplary representation of an aneurysm is provided. A primary vessel 34, an aneurysm sac 36 and an adjacent vessel 38 are shown. Directional arrows depict blood flowing through the primary vessel 34 into both the aneurysm sac 36 and the adjacent vessel 38 (FIG. 7A). Deployment of the device 10 at the site of the aneurysm prevents blood from flowing into the aneurysm sac 36 (FIG. 7B). Of particular note, the adjacent vessel 38 opening is directly across from the aneurysm sac 36 opening. In this example the device 10 has a non-circumferentially placed patch i.e., a patch that covers less, preferably much less, than a 360° completely circumferential fluid-flow restrictive patch or zone, on the scaffold 12, thereby allowing the scaffold 12 to be placed over the adjacent vessel 38 opening with limited disruption of fluid flowing into the adjacent vessel 38 or vessels. Non-circumferential herein means that less than 360° of the inside or outside of the scaffold 12, as the case may be, is covered by the fluid-restrictive patch 14. The degree of circumferential coverage is determined by the radial width of the aneurysm opening which is to be fluid restricted. The frame of reference for determination of the degree of circumferential blockage is from inside the parent vessel. Thus, by example, 30°, 60°, 90° and upwards to just short of 360° subtended scaffold radial circumference, e.g., a large aneurysm opening, are contemplated by the invention.

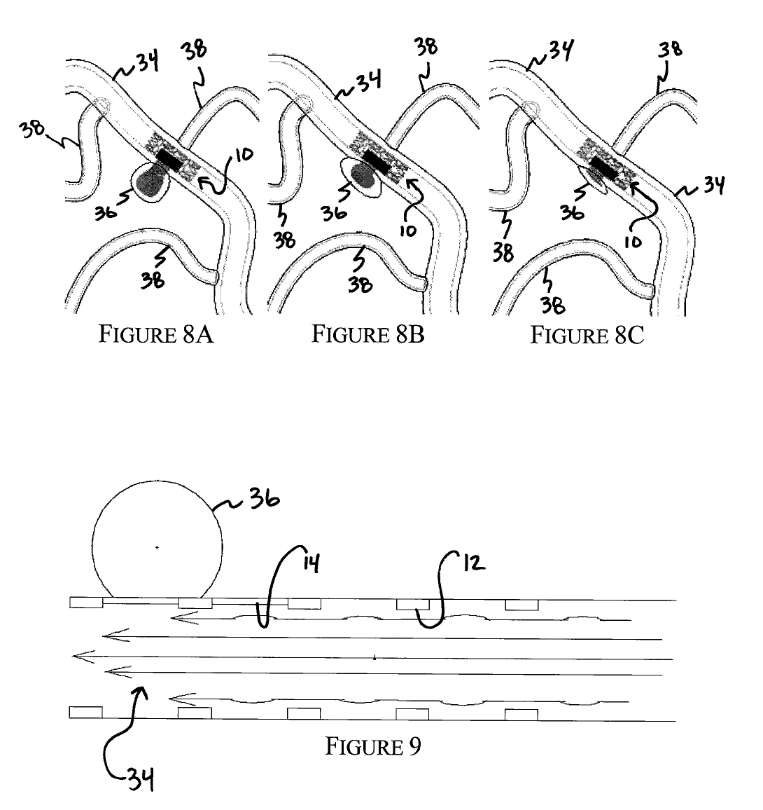

Now referring to FIGS. 8A-8C, an exemplary physiological progression of an aneurysm sac 36 after deployment of the device 10 is depicted. The figures show an exemplary representation of an aneurysm sac or vessel bulge 36 being treated by an SOD 10 and how the aneurysm could react to deployment of the SOD 10. The SOD 10 is deployed inside a cerebral artery 34. Shortly after deployment (FIG. 8A) the aneurysm sac 36 is at its largest and acute state. FIG. 8B represents an intermediate time period post deployment, where the sac 36 has reduced in size due to a lack of blood flow to the aneurysm. As time continues to pass, the degradation of the clot within the sac 36 combined with the remodeling of the aneurysm wall is nearly complete and further reduction of the aneurysm sac 36 occurs (FIG. 8C).

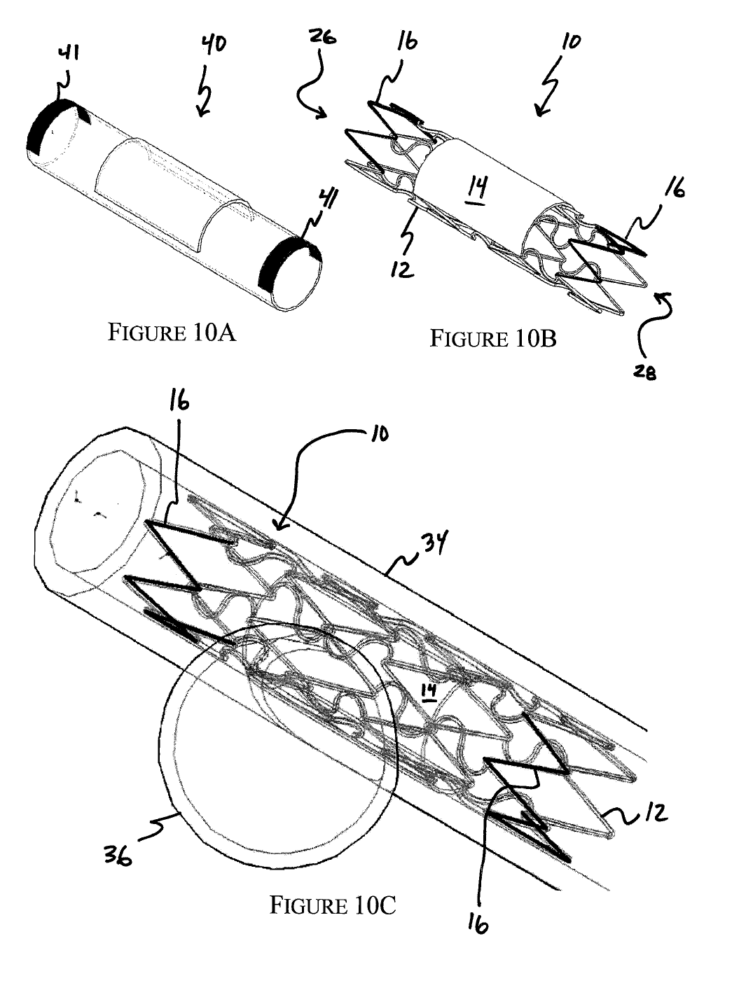

Deployment of the device 10 provides treatment of the aneurysm without blocking fluid flow to an adjacent vessel 38. The radiopaque markers 16 placed at the distal and proximal ends of the frame (see FIGS. 1A, 3A, 4A, 5A and 6) allow a physician to denote axial location of the device 10 (and thusly patch 12) in relation to the aneurysm sac 36. This is of particular importance when the device is intracranially deployed. Unwanted restriction of blood flow within intracranial vessels can have significantly devastating effects upon a patient, and therefore proper placement of the device 10 is critical. A side view of a deployed device 10 is provided in FIG. 9, which graphically represents the minimal disruption of blood flow due to a low-profile device 10. The patch 22 of device 10 (See FIG. 5A) is positioned so as to restrict the flow of fluid into the sac 36 and minimally restricting flow through the vessel 34.

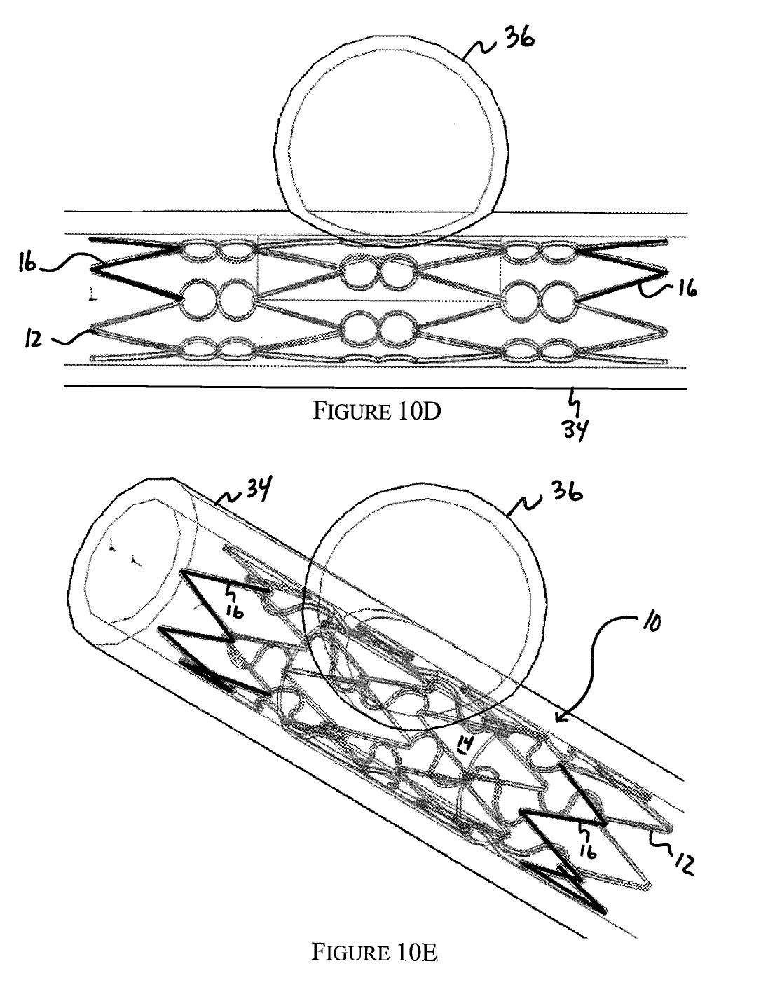

Now referring to FIGS. 10-17, several embodiments of the device 10 are depicted with various radiopaque marker 16 configurations. The markers 16 provide guidance to a physician when deploying the device 10, thereby allowing for correct rotational positioning of the membrane 14. A medical professional, such as a physician, can view the device through a fluoroscope. Preferred orientation of the fluoroscope is often based upon the particular physician's preference. However, one contemplated useful fluoroscope orientation is represented in FIG. 10D.

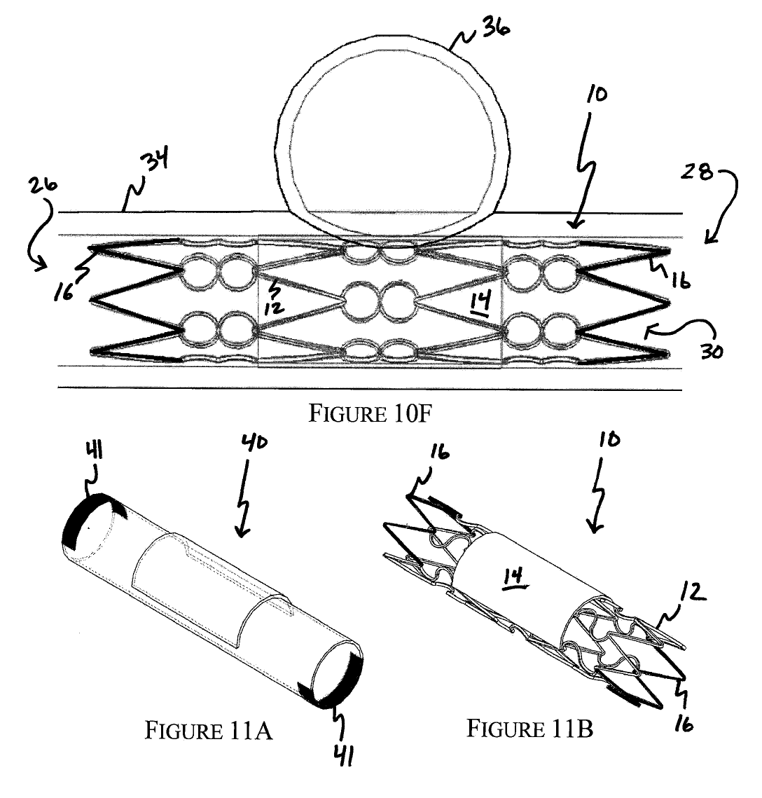

Referring to FIGS. 10A-F, an alternative fluoroscopic marker configuration is provided. An implant frame 40 (FIG. 10A) is provided with a rotationally sensitive pattern representative of the marker 16 configuration on the scaffold 12 (see FIG. 10B). Markers 41 denote the rotationally sensitive pattern. As the device 10 is rotated the length of the marker will either get longer or shorter depending on the orientation. Assuming the physician has a profile view of the aneurysm, the suggested ideal orientation would be when the markers 16 are viewed at their shortest and on the same side as the aneurysm. A corresponding aneurysm treatment device 10 is provided (see FIG. 10B). The present embodiment has a similar marker 16 pattern as compared to FIG. 1A, but the markers 16 are positioned on the top side 30 of the device 10 forming a 180 degree arc. One arc 16 surrounds half the distal end 28 segment 18 circumference while a proximal arc 16 surrounds the other half of the circumference. FIGS. 10C and 10D depict the correct placement of the device 10 where the patch 14 is positioned across the aneurysm neck, thereby inhibiting fluid flowing into the aneurysm sac 36. The membrane is incorrectly positioned away from the aneurysm (see FIGS. 10E and 10F) if the device is rotated. Alternatively, FIG. 11B represents a radiopaque pattern on the implant frame 12 corresponding to a device 10 with an alternative radiopaque pattern (see FIGS. 1A and 11B)

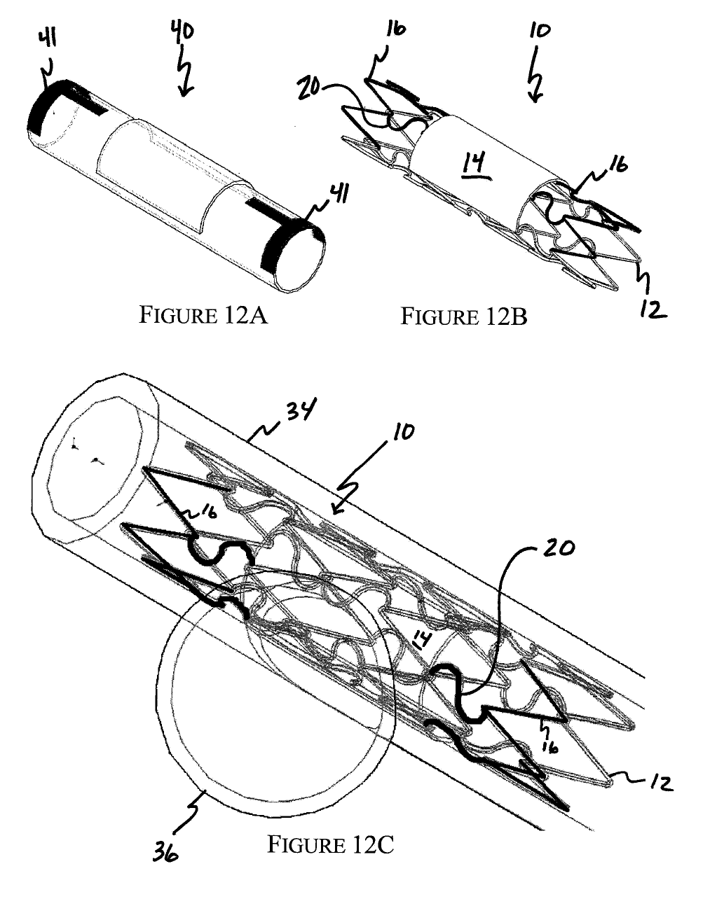

Referring to FIGS. 12A-12E, yet another alternative radiopaque pattern is provided on the aneurysm treatment device 10. The implant frame 40 generally has a T-shaped pattern running along the distal and proximal ends 26, 28, and the bottom of the T-shape extends on the long axis of the frame 40. Both distal and proximal markers 41 are located at the top side 30 of the frame 40. The shape of the T will indicate the position of the patch 14 in regards to its rotational position. The corresponding radiopaque pattern on the scaffold 12 is the same as FIG. 10B, with the additional marking of two segments 20 (see FIG. 12B) proximal to the top side 30. Correct in-situ placement of the device 10 is depicted in FIGS. 12C and 12D. Incorrect in-situ placement of the device 10 is depicted in FIG. 12E.

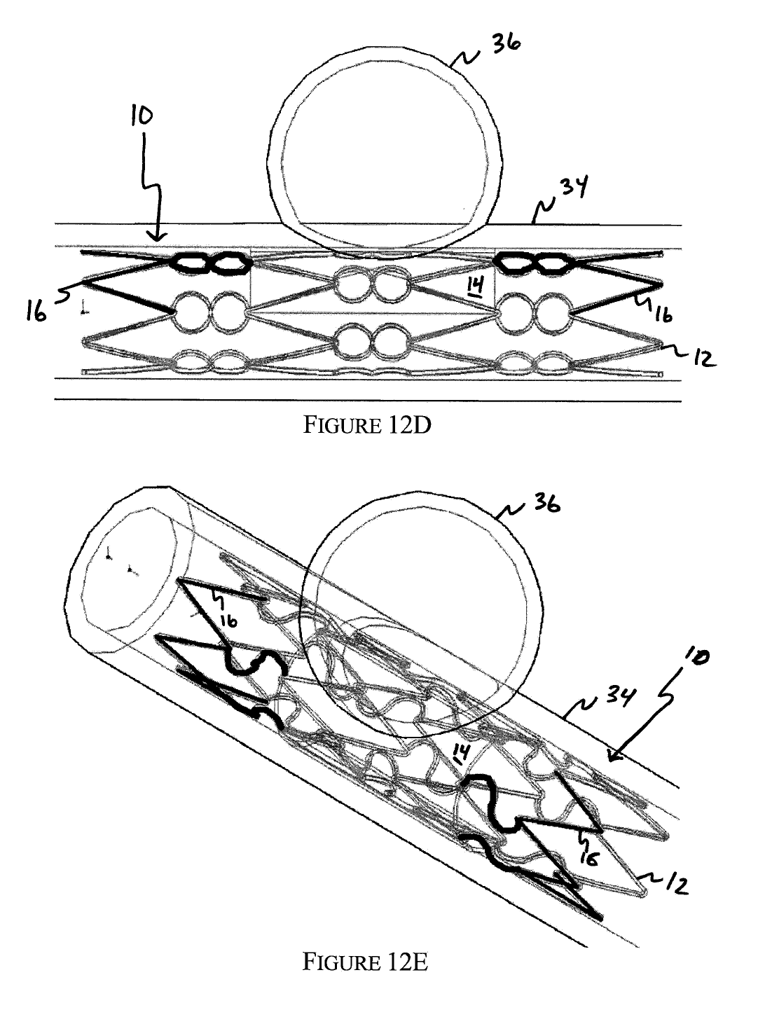

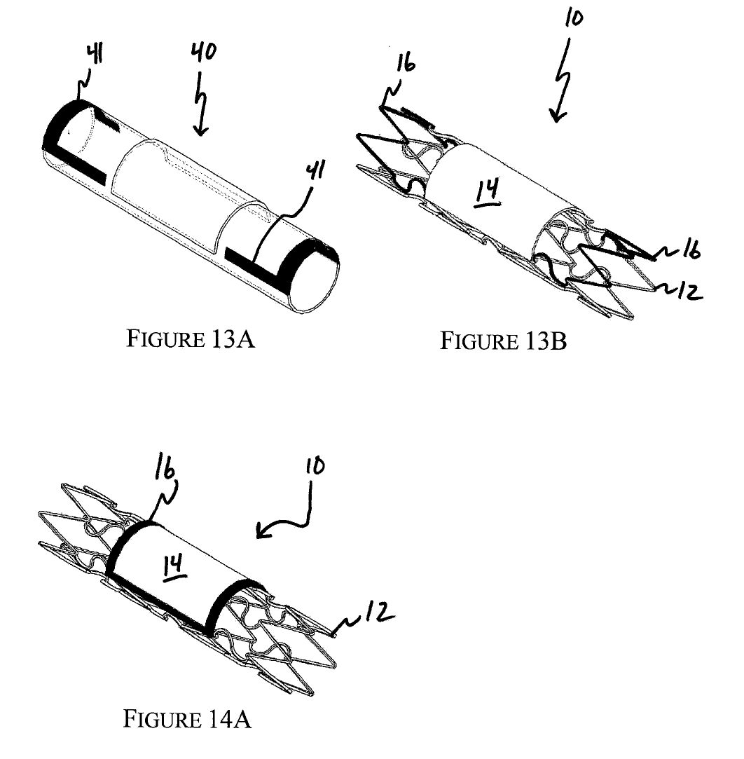

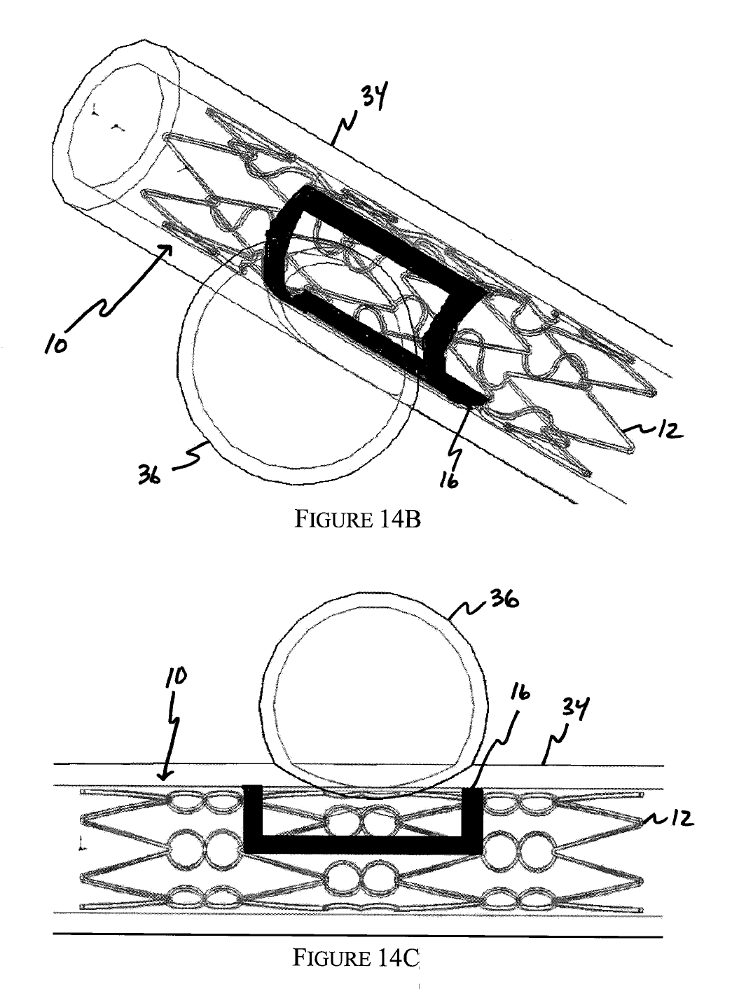

Referring to FIGS. 13A-13B, an alternative radiopaque pattern is provided. The pattern generally has a U-shape. The scaffold 12 has the same pattern as FIG. 10B, with the additional marking of two segments 20 (see FIG. 12B) on opposing sides of the device 10. Alternatively, the radiopaque marker 16 is disposed on the patch 14. Various patterns are contemplated, including an outline of the patch 14 (see FIG. 14A). Correct in-situ placement of the embodiment shown in FIG. 14A is depicted in FIGS. 14B and 14C. In yet another alternative embodiment, the entire patch 14 is embedded with a radiopaque material (See FIG. 15)

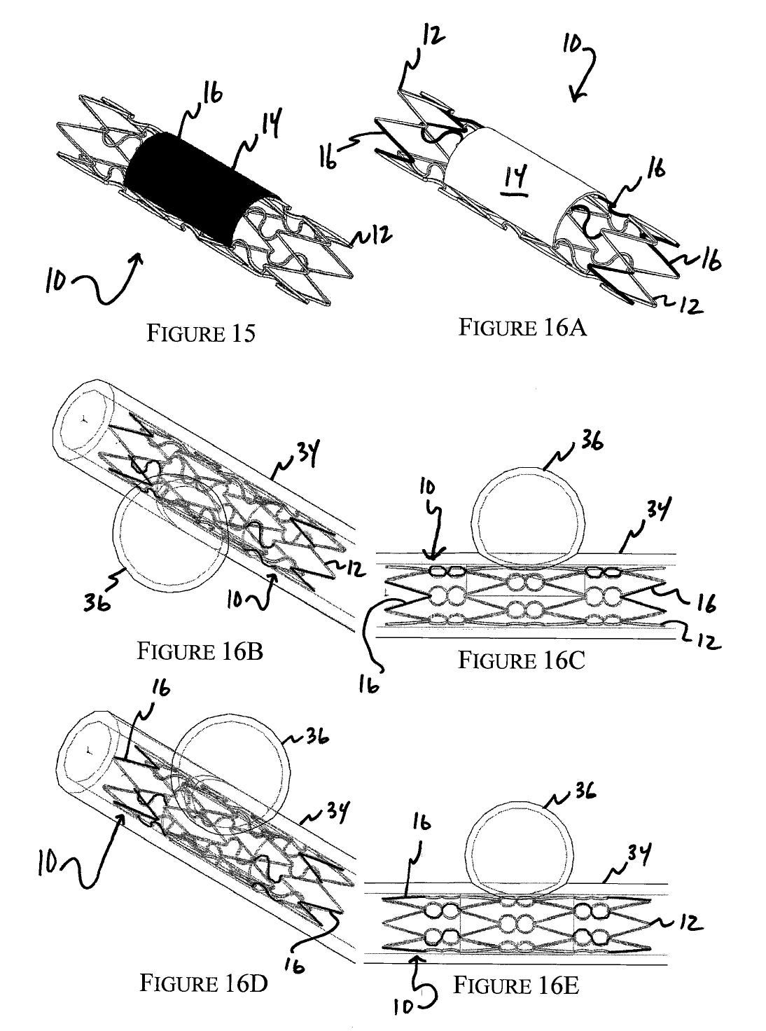

In yet another alternative embodiment, the device 10 has an alternative radiopaque pattern as shown in FIG. 16A. Proper in-situ placement of the device is depicted in FIG. 16B and 16C. Upon additional rotation of the device 10, it is incorrectly placed; the in-situ depiction is represented in FIGS. 16D and 16E.

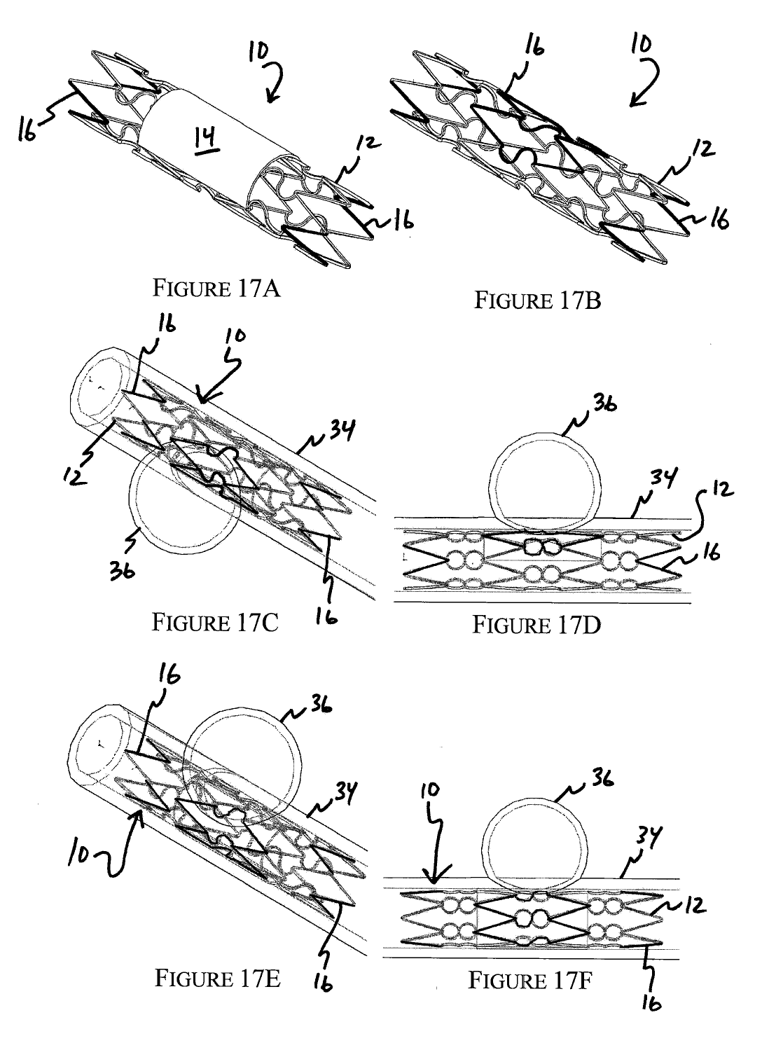

Referring to FIGS. 17A-F, an alternative embodiment of the device 10 is shown. The corresponding radiopaque pattern is shown on the scaffold 12 in FIG. 17B. This particular pattern differs from the previous embodiments in that a portion of the pattern is generally located on the scaffold 12 proximal to where the patch 14 is placed. Additionally, the shape of the pattern corresponding to the patch 14 has a similar shape as the patch 14 in FIG. 6. It is further contemplated that the patch 14 of FIG. 6 is embedded with a radiopaque material (FIG. 15). Proper in-situ placement of the device 10 in FIG. 17A-B is generally depicted in FIGS. 17C and 17D. Upon rotation of the device 10 as shown in 17C, it is incorrectly placed; the in-situ depiction is represented in FIGS. 17E and 17F.

Each of the radiopaque patterns described above represent markers 16 which indicate the axial position of the aneurysm treatment device 10, as well as the rotational orientation of the membrane component. The aneurysm treatment device 10 is partially deployed to see the radiopaque pattern on the distal portion, rotated to correctly orient the membrane, then fully deployed. After full deployment, the radiopaque pattern on the proximal end will be visible as well and serve as a check on the rotational position. A medical professional knowing the location of markers 16 relative to patch 14 can observe the markers 16 while deploying the device 10 and thereby ensure that patch 14 is placed so as to restrict body fluid flow into an aneurysm bulge, bulb, or other sac-like structure and to permit the sac-like structure to heal. Note that various embodiments of the present invention permit aneurysm healing or reabsorbtion without a need to place polymers, metal or any other foreign material into the bulge or sac 36 of the aneurysm.

It is further contemplated that alternative radiopaque patterns are suitable for denoting in-situ rotational orientation, such that a physician can properly place the device 10 within the vasculature based upon the radiopaque pattern. Although various embodiments have been described and depicted with respect to a preferred scaffold configuration, it is contemplated that alternative scaffold 12 configurations are suitable as described herein. Radiopaque patterns applied to alternative scaffold 12 and patch 14 configurations that denote in-situ rotational orientation are also contemplated. One skilled in the art will also understand that other radiopaque patterns exist and can be applied to the SOD frame and/or membrane to denote rotational orientation of the membrane.

The treatment device 10 can be delivered to the site of an aneurysm in a variety of methods. A preferred method of delivery includes a torque wire-based delivery system. Use of a torque wire capable of translating proximal rotation and insertion forces reliably and predictably into distal rotation and displacement at the aneurysm site is one device delivery approach. A mechanical lock between the device and torque wire, or an adhesive coating/foam on the wire tacky enough to rotate a scaffold compressed into it, but allows the scaffold to detach when deployed past the catheter tip, are other approaches.

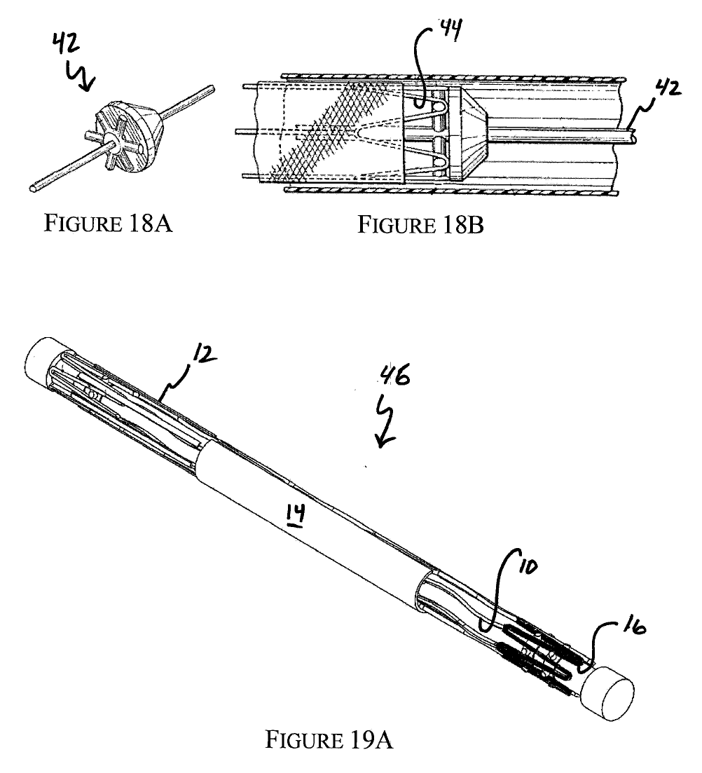

Referring to FIG. 18A, a representative torque-based guide wire interface 42 is provided. A cross sectional view of the interface 42 in connection with a scaffold 44 is shown in FIG. 18B. The interface system is described in U.S. Pat. No. 6,077,297, which is hereby incorporated by reference in its entirety herein.

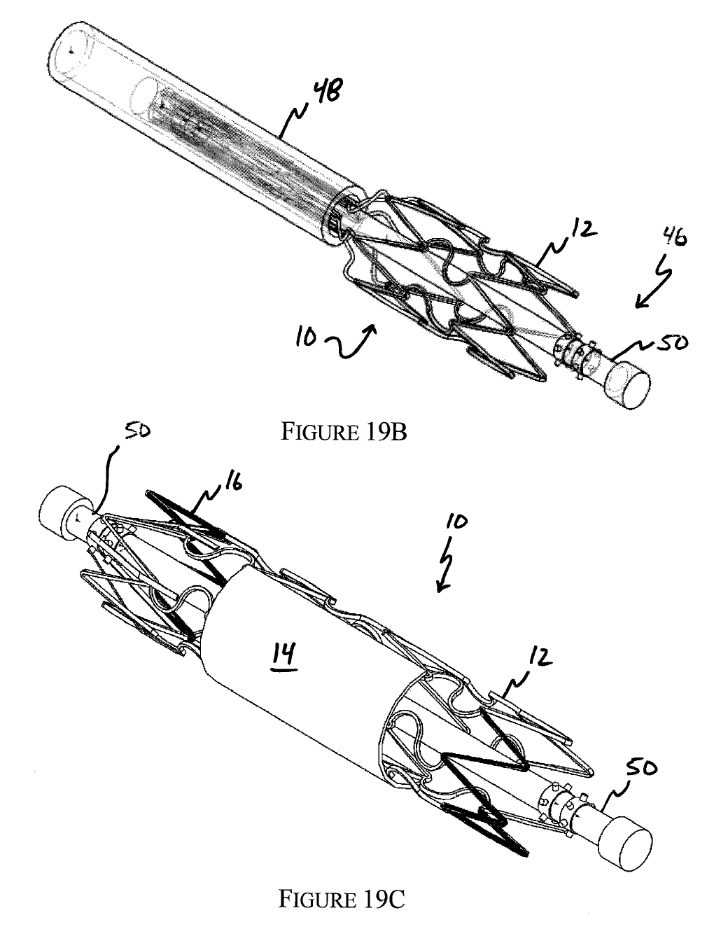

Referring to FIG. 19A, an alternative embodiment of a torque-based guide wire interface 46 having a mechanical interlock is provided. The interface 46 includes a compressed SOD 10 in a configuration prior to deployment. FIG. 19B represents a partially deployed SOD 10 extending from a delivery sleeve 48 and crimped onto a delivery wire 50. The SOD 10 constrained radially by the delivery sleeve 48.

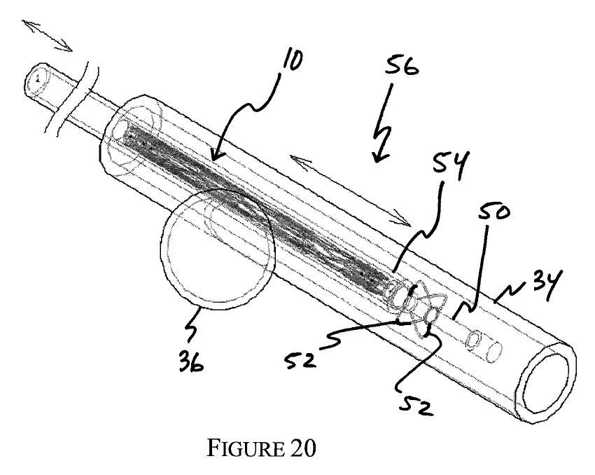

Referring to FIG. 20, a means of identifying the rotational translational position of the patch 14 with respect to an aneurysm 36 is to implement an indicator(s) 52 distal to the device 10 on the same delivery wire 50 that is controlling the SOD 10. The pilot indicators 52 are positioned such that the orientation of the SOD 10 can be determined by the orientation of the pilot indicators 52. The wire 50 will have markers 52 on it that will be deployed ahead of the SOD 10. The indicator 52 will expand to less than the inside diameter of the vessel 34. This will allow free rotation of the device 10 while still allowing better resolution of the markers 52 (as compared to the crimped position). While the indicators 52 are outside of the delivery catheter 54, the SOD 10 is still constrained within the delivery catheter 54. The physician will rotate the markers 52 in reference to the aneurysm 36 so that when the SOD 10 is deployed, the patch 14 will cover the aneurysm neck within the vessel 34.

In this case once the delivery system 56 is in position, having been advanced within the cerebral vasculature 34 and positioned distally to an aneurysm 36 the following steps are performed.
A) The micro-catheter 54 is pulled back to deploy the pilot indicator 56 portion of the delivery wire 50.
B) The entire system 56 (including microcatheter, delivery wire and SOD) will be moved longitudinally until the SOD 10 is in the same location as on the aneurysm 36, which is guided by the pilot indicator 52.
C) The delivery wire 50 will be rotated in the direction of the aneurysm 36 as directed by the indicator 52 to properly locate the patch 14 in front of the aneurysm 36.
D) If the position is satisfactory, then the microcatheter 54 is pulled back against the delivery wire 50 to partially deploy the SOD 10.
E) Rotational and axial adjustments are made based upon the position of the device markers 52.
F) In the event that adjustments do not result in satisfactory positioning of the device 10, then the device 10 is recaptured, which includes stationary placement of the delivery wire 50 followed by advancement of the catheter 54. Steps D and E are repeated until the physician is satisfied with the position of the partially deployed device 10.
E) After satisfactory placement of the device 10 occurs, the microcatheter 54 is pulled back in order for the device 10 to be fully deployed.
F) The microcatheter 54, delivery wire 50, and indicator 52 are withdrawn.

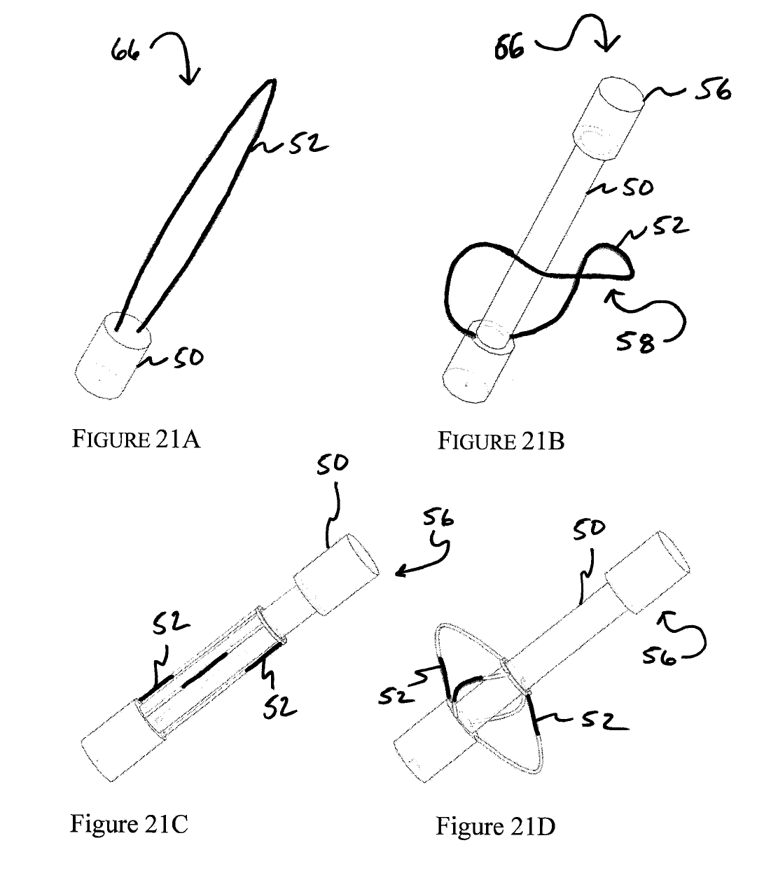

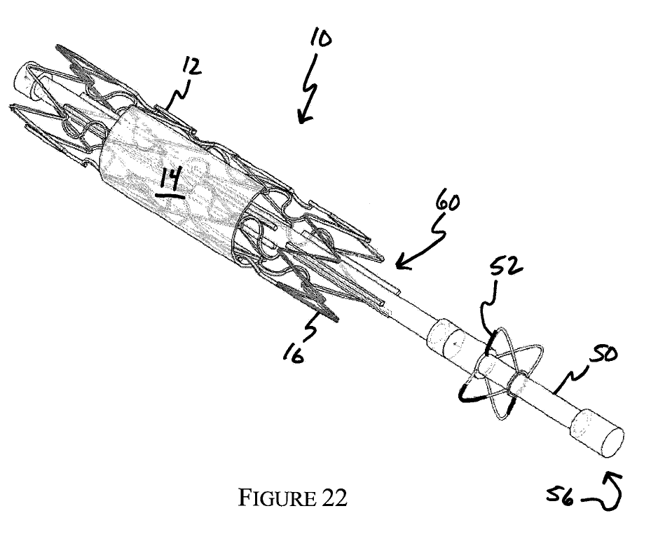

Now referring to FIGS. 21A-D, various embodiments of the pilot indicator 52 are shown. FIG. 21A includes the distal end 66 of a delivery wire having a generally pointed pilot indicator 52. FIG. 21B includes a distal end 66 of an alternative configuration for both delivery wire 50 and pilot indicator 52 in the deployed position. The delivery wire 50 has a distal tip 56, which extends beyond a bent pilot indicator configuration 58. FIGS. 21C and 21D include the same delivery wire configuration as 21B with an alternative configuration for the pilot indicator 52. FIG. 22 includes a combination of the delivery wire 50, an embodiment of a mechanical interlock mechanism 60, device 10, and pilot indicator 52 of FIG. 21D, all shown in a deployed or expanded configuration.

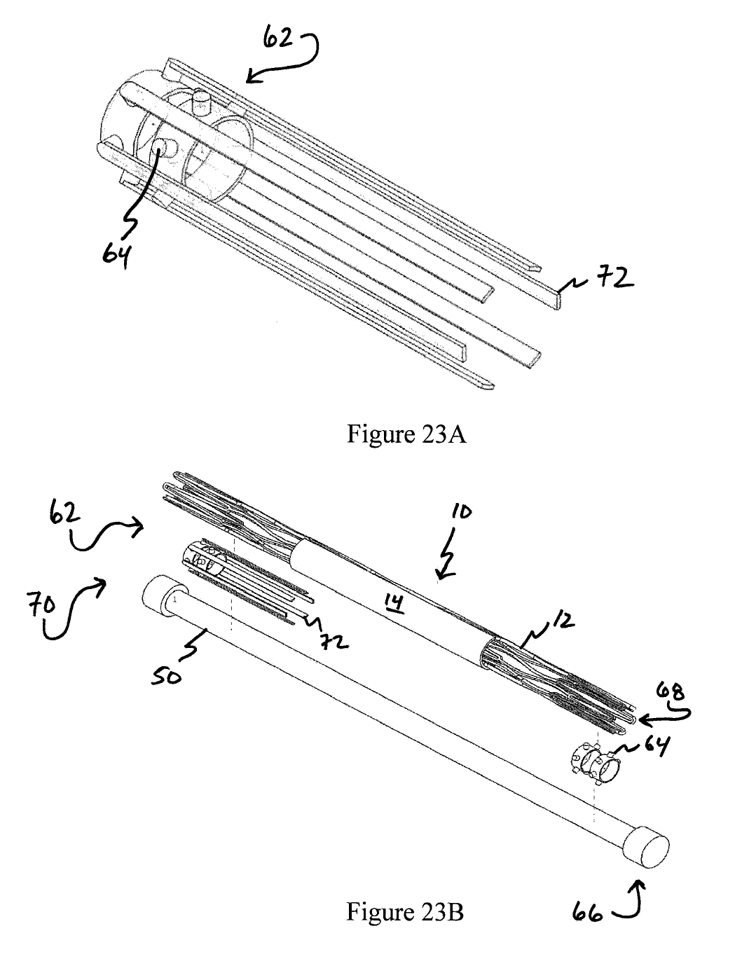

An alternative embodiment of the delivery wire and device interlock mechanism 62 is shown in FIGS. 23A and 23B. An alternative mechanical interlock mechanism is shown in FIGS. 18A and 18B, which includes a plurality of spokes that fit in between struts. The spokes prevent premature deployment as the stent is pushed out of the catheter. This mechanical interlock configuration is an alternative means for axially and rotationally positioning the device 10 within the vasculature.

FIGS. 23A and 23B represent an adaptation of a spoke design. By having a complimentary pair of spokes 64 at a distal end 66, the struts 68 are locked tightly, allowing for rotation of the delivery wire 50 to translate into rotation of the device 10. On the proximal end 70, struts are sandwiched between a set of spokes 64 and a set of spoke with extensions 72. A very small portion of the extensions 72 remain in contact with a fully expanding frame 12, allowing for some minor final adjustments to rotational orientation before the delivery wire 50 is retrieved.

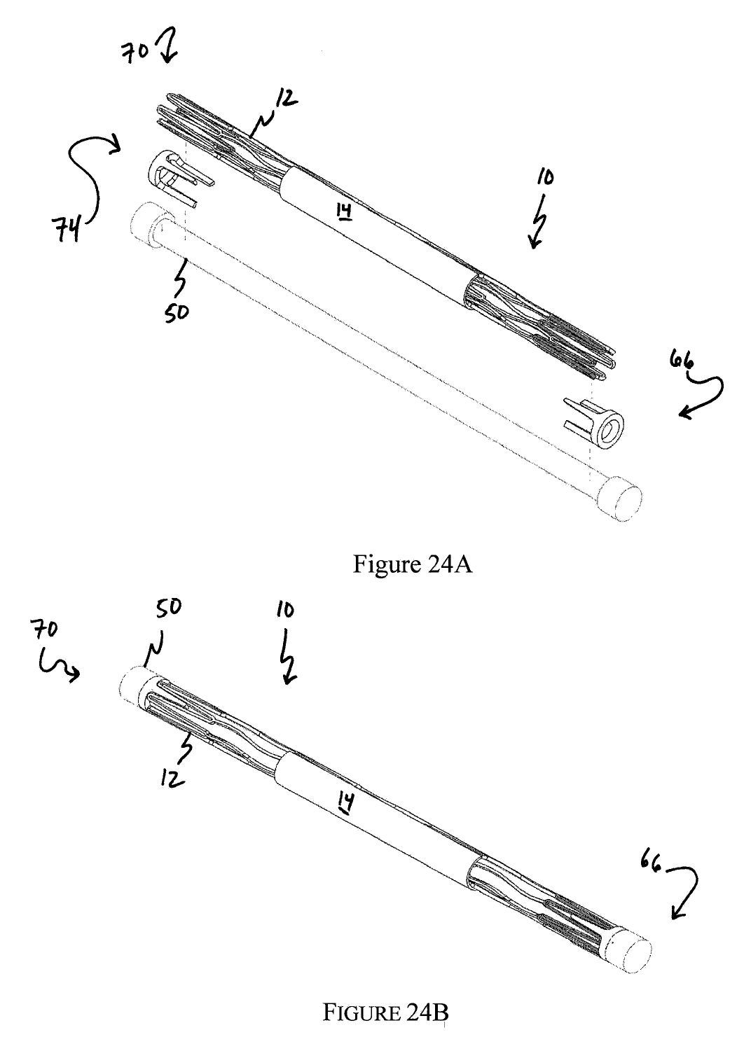

An alternative embodiment of the delivery wire 50 and device interlock mechanism 74 is shown in FIGS. 24A and 24B. The present embodiment is a simpler mechanical interlock design 74 that is complimentary to the struts at the proximal 70 and distal ends 66 of the scaffold 12. Much like the embodiment described in FIGS. 23A and 23B, the interlock locks in tightly with the ends of the scaffold 12, translating any rotation of the delivery wire 50 to rotation of the scaffold 12. Alternative means for positioning the device 10 are contemplated, which can include delivery wires that have adhesive, pliable, sintered, or roughened surfaces that allow rotational movement of the pre-deployed device through friction. Additional means for positioning the device 10 allow for rotational and axially movement of the device 10.

In an alternative embodiment, the markers 16 can be echogenic and therefore ultrasonically visible. The echogenic markers 16 can be in the form of a coating, on either patch 14, scaffold 12, or a combination of both patch 14 and scaffold 12. All radiopaque marker 16 configurations described herein are contemplated and suitable as echogenic markers 16. Furthermore, a means for identifying the rotational translational position of the patch 14 in which echogenic markers are employed, can be performed with the use of an ultrasound device, such as a medical sonographic machine or other ultrasound-based diagnostic machines known in the art. Additionally, the scaffold 12 and/or the patch 14 can be manufactured, at least partially, from an echogenic material, and therefore the markers 16 are embedded within the structure of the scaffold 12 and/or patch 14. It is also contemplated that the pilot indicator 52 has an echogenic coating or is manufactured from at least partially an echogenic material. Exemplary echogenic coatings are described within U.S. Pat. No. 7,229,413, issued Jun. 12, 2007, hereby incorporated by reference in its entirety herein. Additional echogenic coatings and materials are contemplated, which are known in the art, and suitable for visualizing the axial and rotational positioning of the device 10 within the vasculature.

It is specifically intended that the present invention not be limited to the embodiments and illustrations contained herein, but rather that the present invention also include modified forms of those embodiments including portions of the embodiments and combinations of elements of different embodiments as come within the scope of the following claims.

What is claimed is as follows:

1. A cylindrical intravascular aneurysm treatment device comprising:
an expandable sleeve, the sleeve having,
a fluid-flow-restricting patch, the patch being located on the sleeve so as to inhibit fluid flow through a portion of the sleeve when the sleeve is intravascularly deployed, the device further comprising;
a radiopaque marker disposed on the sleeve, the marker having a defined relationship with respect to the patch such that deployment of the sleeve within the vasculature determines the location of the patch.

2. The device according to claim 1, wherein the sleeve is flexible.

3. The device according to claim 1, wherein the sleeve is retractable and repositionable.

4. The device according to claim 1, wherein the patch is comprised of a material selected from the group consisting of a polymeric or biostable material selected from the group consisting of parylene, polysiloxane, silicone, polyurethane and ePTFE.

5. The device according to claim 1, wherein the patch is partially circumferentially and axially disposed on the sleeve.

6. The device according to claim 1, wherein the patch is disposed on an outside portion of the sleeve.

7. The device according to claim 1, wherein the patch is disposed on an interior portion of the sleeve.

8. The device according to claim 1, wherein the patch is integrally formed with the sleeve.

9. The device according to claim 1, wherein the marker is disposed on the patch.

10. The device according to claim 1, wherein the device comprises a pair of opposable ends, the marker being disposed at the opposable ends of the patch.

11. The device according to claim 1, wherein the marker is non-symmetrically positioned with respect to the patch, the patch being configured to restrict fluid flow to a specific vascular region.

12. An intracranial aneurysm treatment device comprising:
an expandable scaffold having,
a fluid-flow-restricting patch, the patch inhibiting fluid flow through a non-circumferential portion of the scaffold, the device further comprising;
a radiopaque marker having a non-symmetrically defined relationship with respect to the patch, the marker configured to be observed during deployment of the scaffold within the cranial vasculature to determine the location of the patch.

13. The device according to claim 12, wherein the patch is an occlusive membrane comprised of a biostable material selected from the group consisting of parylene, polysiloxane, polyurethane and ePTFE.

14. The device according to claim 12, wherein the scaffold is intravascularly-dimensioned subsequent to deployment.

15. The device according to claim 13, wherein the patch is disposed on both of the interior and exterior of the scaffold.

16. The device according to claim 13, wherein the marker is a radiopaque coating partially disposed on the scaffold.

17. The device according to claim 13, wherein the marker is integral to the patch

18. A method of treating an aneurysm comprising the steps of:
a) providing an aneurysm treatment device comprising an expandable scaffold, in combination with a fluid-flow-restricting, non-circumferentially positioned patch and a radiopaque marker having a defined relationship with respect to the patch;
b) deploying the device of step (a) into the vasculature of a patient with a aneurysm while simultaneously monitoring the location of the radiopaque markers to place the patch adjacent the aneurysm in a location which restricts fluid flow thereto; and
c) permitting the aneurysm to constrict based on reduced fluid flow thereto.

19. The method according to claim 18, further comprising the step of partially expanding the scaffold for greater ease of rotational movement within a vasculature prior to deploying the device.

20. The method according to claim 18, wherein deploying the device consists of placing the patch so as to avoid restricting fluid flow to a vessel adjacent the aneurysm.

21. The method according to claim 18, wherein the device is intravascularly-dimensioned after deploying.

22. An aneurysm delivery system comprising:
an aneurysm treatment device comprising an expandable scaffold, in combination with a fluid-flow-restricting, non-circumferentially positioned patch and a marker having a defined relationship with respect to the patch; and means for deploying the aneurysm treatment device into a patent's vasculature so that fluid flow to the aneurysm is restricted.

23. The system according to claim 22, wherein the means for deploying the device is a mechanical interlock attached to a torque-able guidewire, the interlock configured to allow rotational and axial positioning of the device within a vessel.

24. The system according to claim 22, wherein the means for deploying the device is an adhesive or pliable coating disposed on a guidewire, the adhesive or pliable coating configured to allow rotational and axial positioning of the device within a vessel.

25. The system according to claim 22, wherein the marker is radiopaque.

26. The system according to claim 22, wherein the marker is echogenic.

27. The system according to claim 26, wherein the patch is echogenic.

28. A cylindrical intravascular aneurysm treatment device comprising:
an expandable sleeve, the sleeve having,
a fluid-flow-restricting patch, the patch being located on the sleeve so as to inhibit fluid flow through a portion of the sleeve when the sleeve is intravascularly deployed, the device further comprising;
an echogenic marker disposed on the sleeve, the marker having a defined relationship with respect to the patch such that deployment of the sleeve within the vasculature determines the location of the patch.

29. The device according to claim 28, further comprising a delivery wire having a pilot indicator positioned proximal to a tip of the delivery wire, the pilot indicator having a defined relationship with respect to the patch such that placement of the pilot indicator determines the approximate location and orientation of the patch.
